(19) United States
(12) Patent Application Publication
(10) Pub. No.: US 2007/0129681 A1
(43) Pub. Date: Jun. 7, 2007

(54) PIEZOELECTRIC ACTUATION OF PISTON WITHIN DISPENSING CHAMBER

(75) Inventor: William F. Ott, Willaimsburg, VA (US)

Correspondence Address: NIXON & VANDERHYE, PC, 901 NORTH GLEBE ROAD, 11TH FLOOR, ARLINGTON, VA, 22203, US

(73) Assignee: PAR Technologies, LLC, Hampton, VA (US)

(21) Appl. No.: 11/555,343

(22) Filed: Nov. 1, 2006

Related U.S. Application Data
(60) Provisional application No. 60/723,480, filed on Nov. 1, 2005.

(51) Int. Cl. A61M 5/00; A61M 1/00

(52) U.S. Cl. 604/152; 604/207

(57) ABSTRACT

A fluid dispenser (20) comprises a housing (22) for defining a fluid chamber (24). The housing (22) has an orifice (26) through which fluid is discharged. A piston (28) is positioned in the housing (22) for linear motion in the chamber for expelling fluid from the chamber and through the orifice (26). A piezoelectric actuator assembly (30) is positioned in the housing for imparting the linear motion to the piston. In one example implementation, the fluid dispenser housing takes the form of a syringe. In the syringe implementation, a syringe housing defines an essentially cylindrical fluid chamber. In an example embodiment, the piezoelectric actuator assembly (30(4)) comprises a first piezoelectric actuator (50₁); a second piezoelectric actuator (50₂); and, a circuit (34(4)) for actuating the first piezoelectric actuator (50₁) and the second piezoelectric actuator (50₂).

This application claims the benefit and priority of U.S. Provisional Patent Application 60/723,480, filed Nov. 1, 2005, entitled “PIEZOELECTRIC ACTUATION OF PISTON WITHIN DISPENSING CHAMBER”, which is incorporated herein by reference in its entirety.

### BACKGROUND

1. Field of the Invention

The present invention pertains to fluidic dispensing devices, and particularly to a fluidic dispensing device capable of dispensing fluid in fine increments or dosages.

2. Related Art and Other Considerations

In myriad environments fluids are delivered or dispensed in controlled manner from disposable, inexpensive containers (e.g., bags, pouches, cartons, cartridges, just to name a few). The dispensing may be controlled to obtain a required or target dosage or amount over time, such as (for example) control of a medicament to a patient or an ingredient utilized in an industrial or other process. Typically such control is achieved by an actuator which is external to the disposable fluid container. The actuator is generally considerably more expensive than the disposable fluid container, and thus for sake of practicality must be provided externally so that it can be reused.

What is needed, and an object of the present invention, is a fluid dispensing device which has an integrated actuator capable of dispensing fluid in fine increments or dosages. Advantages are that the fluid dispensing device including its integrated actuator is disposable and inexpensive.

### BRIEF SUMMARY

A fluid dispenser comprises a housing for defining a fluid chamber. The housing has an orifice through which fluid is discharged. A piston is positioned in the housing for linear motion in the chamber for expelling fluid from the chamber and through the orifice. A piezoelectric actuator assembly is positioned in the housing for imparting the linear motion to the piston.

In one example implementation, the fluid dispenser housing takes the form of a syringe. In the syringe implementation, a syringe housing defines an essentially cylindrical fluid chamber.

In an example embodiment, the piezoelectric actuator assembly comprises a first piezoelectric actuator; a second piezoelectric actuator; and, a circuit for actuating the first piezoelectric actuator and the second piezoelectric actuator. The circuit applies activating signals to the first piezoelectric actuator and the second piezoelectric actuator whereby the first piezoelectric actuator and the second piezoelectric actuator transition between states or positions for respectively contacting a wall of the housing and deflecting in a sequence or pattern whereby the piezoelectric actuator assembly and the piston coupled thereon linearly travel through the chamber for expelling fluid from the chamber and through the orifice.

### BRIEF DESCRIPTION OF THE DRAWINGS

The foregoing and other objects, features, and advantages of the invention will be apparent from the following more particular description of preferred embodiments as illustrated in the accompanying drawings in which reference characters refer to the same parts throughout the various views. The drawings are not necessarily to scale, emphasis instead being placed upon illustrating the principles of the invention.

FIG. 1A is a sectioned side view of a fluid dispensing device according to an example embodiment, showing a piston and integrated piezoelectric actuator in a first position.

FIG. 1B is a sectioned side view of the fluid dispensing device of FIG. 1A but showing the piston and integrated piezoelectric actuator in a subsequent or second position.

FIG. 2 is a sectioned side view of a fluid dispensing device according to an another example embodiment wherein a piezoelectric activation circuit is external to a dispensing device housing.

FIG. 3 is an isometric exploded view of a fluid dispenser in which a fluid dispenser housing takes the form of a syringe.

FIG. 4A-FIG. 4E are sectioned side views of a fluid dispensing device according to another example embodiment, and particularly an actuator assembly having a piezoelectric actuator assembly comprising dual piezoelectric actuator members, and showing respective piezoelectric actuator assembly configurations involved in expelling fluid from the fluid dispensing device.

### DETAILED DESCRIPTION OF THE DRAWINGS

In the following description, for purposes of explanation and not limitation, specific details are set forth such as particular architectures, interfaces, techniques, etc. in order to provide a thorough understanding of the present invention. However, it will be apparent to those skilled in the art that the present invention may be practiced in other embodiments that depart from these specific details. That is, those skilled in the art will be able to devise various arrangements which, although not explicitly described or shown herein, embody the principles of the invention and are included within its spirit and scope. In some instances, detailed descriptions of well-known devices, circuits, and methods are omitted so as not to obscure the description of the present invention with unnecessary detail. All statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Thus, for example, it will be appreciated by those skilled in the art that block diagrams herein can represent conceptual views of illustrative circuitry embodying the principles of the technology. The functions of the various elements including functional blocks labeled as “processors” or “controllers” may be provided through the use of dedicated hardware as well as hardware capable of executing software in association with appropriate software. When provided by a processor, the functions may be provided by a single dedicated processor, by a single shared processor, or by a plurality of individual processors, some of which may be shared or distributed. Moreover, explicit use of the term “processor” or “controller” should not be construed to refer exclusively to hardware capable of executing software, and may include, without limitation, digital signal processor (DSP) hardware, read only memory (ROM) for storing software, random access memory (RAM), and non-volatile storage.

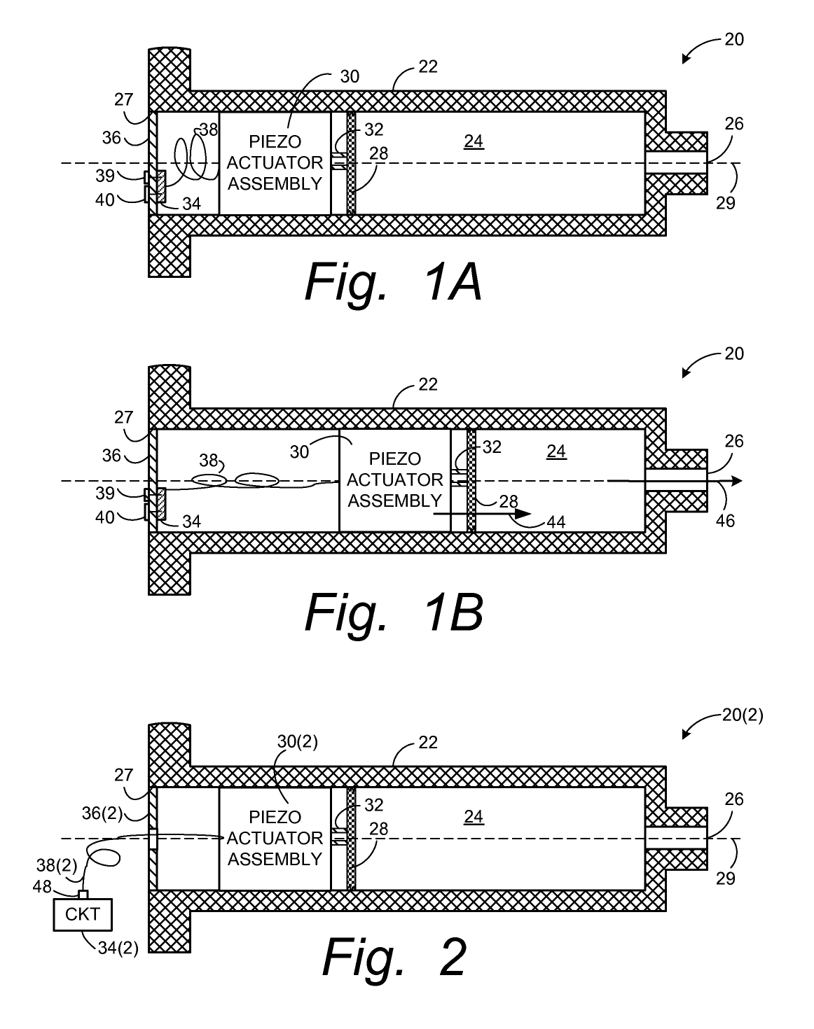

FIG. 1 shows a generic fluid dispenser 20 which comprises a housing 22 for defining a fluid chamber 24. In the example, non-limiting configuration shown in FIG. 1, housing 22 and the fluid chamber 24 provided therein are both essentially cylindrical. The housing 22 has a discharge orifice 26 (through which fluid can be discharged from fluid chamber 24) and a loading port 27. A piston 28 is positioned in housing 22 for linear motion in fluid chamber 24 for expelling fluid from fluid chamber 24 and through orifice 26. In the example, non-limiting configuration shown in FIG. 1, orifice 26 and loading port 27 are positioned at opposite ends of housing 22 with respect to a major cylindrical axis 29. The piston 28 is situated for linear motion along major cylindrical axis 29 toward piston 28.

A piezoelectric actuator assembly 30 is positioned in housing 22 for imparting the linear motion to piston 28. In the illustrated example embodiment, piezoelectric actuator assembly 30 can be coupled to piston 28 by one or more coupling members or struts 32. The piezoelectric actuator assembly 30 comprises one or more piezoelectric actuator members, as well as a piezoelectric activation circuit 34.

Loading port 27 is covered with a lid 36 or closure member lid.The covering of loading port 27 with lid 36 occurs after the desired fluid, the piston 28, and the piezoelectric actuator assembly 30 are inserted (in this order) into fluid chamber 24. In the example, non-limiting configuration shown in FIG. 1, piezoelectric activation circuit 34 is carried by lid 36. In particular, piezoelectric activation circuit 34 is mounted on an underside of lid 36, facing the interior of fluid chamber 24. The piezoelectric activation circuit 34 is connected by one or more electrical leads 38 to piezoelectric actuator assembly 30. If desired, lid 36 can also carry (e.g., on its outerside) an activation switch or button 39 which provides input to piezoelectric activation circuit 34 for, e.g., initiating activity of piezoelectric activation circuit 34. It will also be appreciated that lid 36, or piezoelectric activation circuit 34 itself, may carry one or more batteries or energy supply source for piezoelectric activation circuit 34. The lid 36 may further be provided with an indicator 40 which provides an indication of a state of operation of piezoelectric activation circuit 34 or some other aspect (e.g., identification) of fluid dispenser 20. For example, indicator 40 may take the form of a light emitting diode (LED) or other type of display which indicates that the piezoelectric activation circuit 34 has been activated, or even a display which provides a numeric or other indication of a degree of operation or actuation of piezoelectric actuator assembly 30 (e.g., a number of pulses applied by piezoelectric activation circuit 34 to piezoelectric actuator assembly 30, or an estimated amount of dosage either already applied from or remaining in fluid chamber 24).

The piezoelectric activation circuit 34 of piezoelectric actuator assembly 30 serves to apply electrical signals (e.g., pulses) to the one or more piezoelectric actuators of piezoelectric actuator assembly 30. Piezoelectric materials can be defined by demonstration of the direct piezoelectric effect, which is the ability to polarize under an applied strain. The corollary to this effect is the inverse piezoelectric effect, which is a material's ability to strain under an applied electric field. This physical response to a stimulus is rooted in the displacement of ionic charges within a crystal structure. The PZT (lead zirconate titanate) component is a piezoelectric material, as this class of materials exhibits the piezoelectric effect. Most commercially available PZT materials are polycrystalline, and therefore the displacement of ionic charges takes place in domains where all polarization vectors are aligned. These domains are initially oriented through application of a strong DC field (“poling”), which only partially aligns the dipoles due to their polycrystalline nature. Complete domain alignment is theoretically possible in single crystal PZTs.

The electrical signals applied by piezoelectric activation circuit 34 to piezoelectric actuator assembly 30 cause the piezoelectric actuator(s) comprising piezoelectric actuator assembly 30 to selectively and repeatedly deform and then rebound. For example, if the piezoelectric actuator assembly 30 has an essentially circular or disk configuration, the diameter of the piezoelectric member(s) change(s) in accordance with application of the electrical signals. As such, the piezoelectric member(s) transition between a dome-like deformed state of smaller diameter (in which the edges of piezoelectric actuator assembly 30 do not contact or grip the inside wall of housing 22) and one or more states of larger diameter(s) in which the edges of piezoelectric actuator assembly 30 do contact or grip the inside wall of housing 22. The timing of the electrical signals is controlled in a manner whereby the piezoelectric actuator assembly 30 selectively grips, then reaches along an interior wall of housing 22 further toward orifice 26, and then grips again and reaches again and again. With the repeated gripping and reaching, the piezoelectric actuator assembly 30 and the piston 28 mounted thereto successively crawl toward orifice 26. As piston 28 travels toward orifice 26, fluid is expelled from fluid chamber 24.

FIG. 1B shows the fluid dispenser 20 of FIG. 1A after piezoelectric actuator assembly 30 and piston 28 have traveled or “crawled” further toward orifice 26, as depicted by arrow 44. The travel or crawl of piezoelectric actuator assembly 30 and piston 28 causes expulsion of fluid from fluid chamber 24 through orifice 26, as indicated by arrow 46. The increments and the magnitude of the crawls are timed by the signals applied by piezoelectric activation circuit 34 to piezoelectric actuator assembly 30. Thus, the timing and amount of dosage or discharged fluid from fluid chamber 24 can be precisely metered by piezoelectric activation circuit 34.

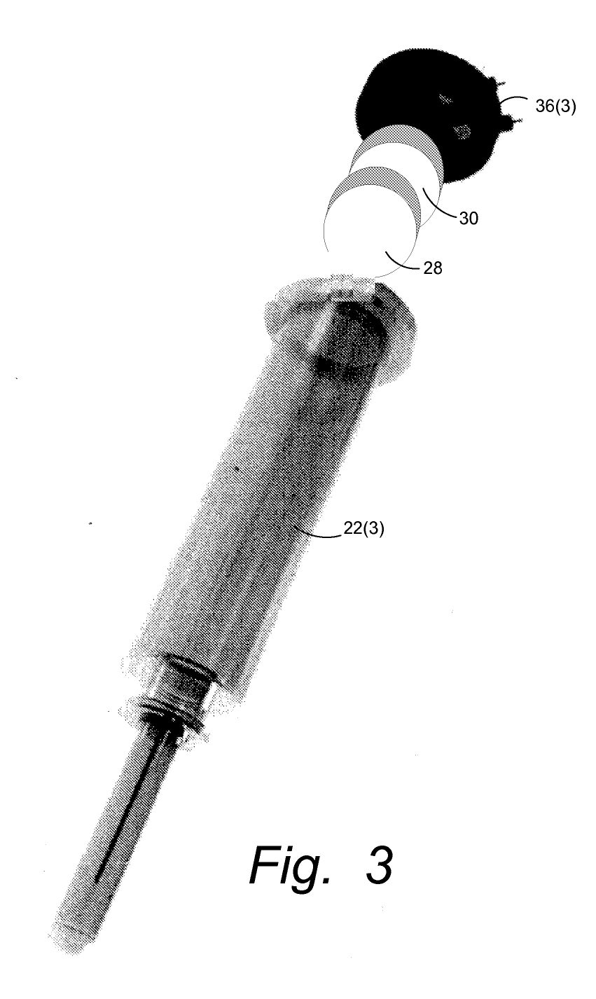

FIG. 2 shows a variation of the fluid dispenser 20 of FIG. 1A and FIG. 1B.In particular, FIG. 2 shows fluid dispenser 20(2) wherein a piezoelectric activation circuit 34(2) of piezoelectric actuator assembly 30(2) is external to housing 22. As such, electrical leads 38 extend through an opening or the like in lid 36(2) and have one or more terminals 48 of electrical leads 38(2) that attach to piezoelectric activation circuit 34(2). In this manner, and if desired, piezoelectric activation circuit 34(2) can be reused for other fluid dispensers.

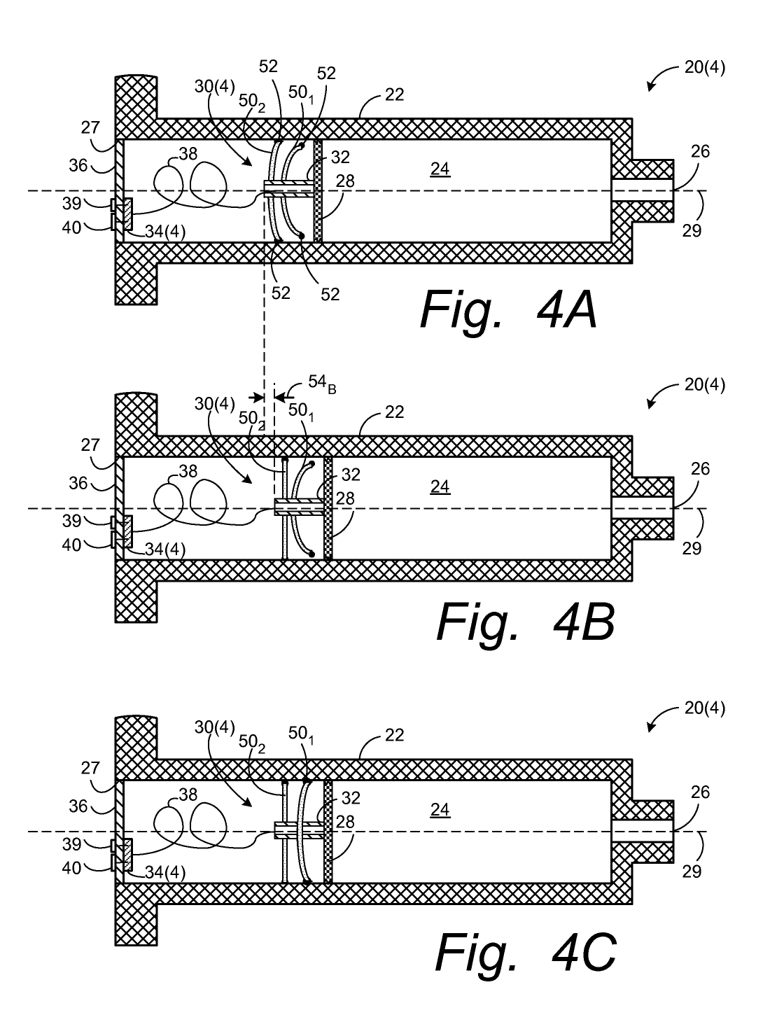

In one example implementation, the fluid dispenser housing 22(3) takes the form of a syringe such as that shown in FIG. 3. In the syringe implementation, the syringe housing 22(3l ) defines an essentially cylindrical fluid chamber. FIG. 3 shows housing 22(3) in an exploded configuration, it being understood that piston 28 with its attached piezoelectric actuator assembly 30 is inserted into the barrel of housing 22(3) after fluid is loaded into fluid chamber 24.

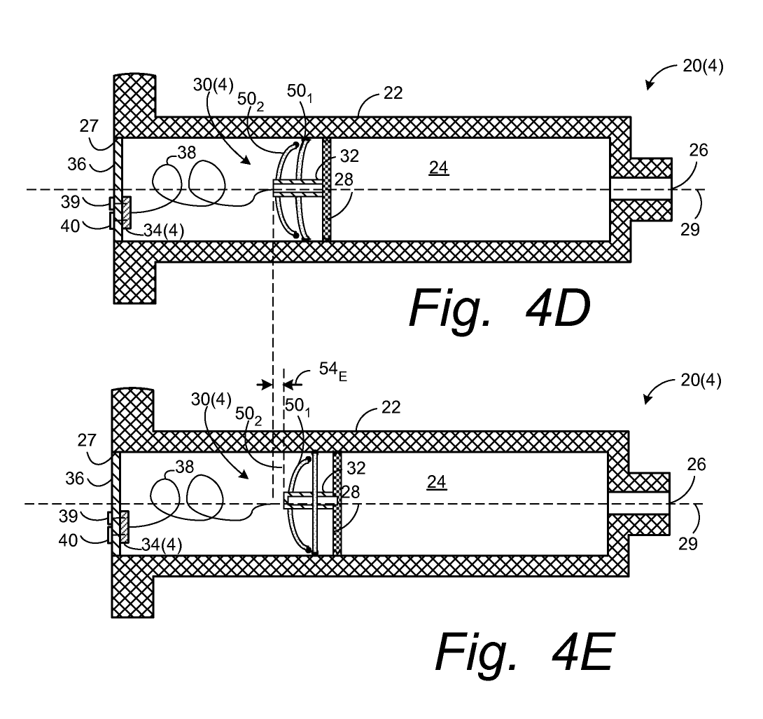

FIG. 4A shows an embodiment of a fluid dispenser 20(4) with an actuator assembly 30(4) having dual piezoelectric actuator members. In particular, the piezoelectric actuator assembly 30(4) of fluid dispenser 20(4) comprises a first piezoelectric actuator 50₁; a second piezoelectric actuator 50₂; and, a circuit 34(4) for actuating the first piezoelectric actuator 50₁ and the second piezoelectric actuator 50₂.

In the example implementation of FIG. 4A, both first piezoelectric actuator 50₁ and second piezoelectric actuator 50₂ are mounted by coupling strut 32 to piston 28. The coupling strut 32 can take the form of a cylinder (preferably hollow) extending between piston 28 and piezoelectric actuator assembly 30(4) along major cylindrical axis 29. The coupling strut 32 is rigidly attached to the side of piston 28 which is not in fluid contact. A distal end of coupling strut 32, which is oriented toward lid 36, extends through apertures centrally formed in first piezoelectric actuator 50₁ and second piezoelectric actuator 50₂. The first piezoelectric actuator 50₁ and second piezoelectric actuator 50₂ are secured in fixed positions on coupling strut 32. Affixation of first piezoelectric actuator 50₁ and second piezoelectric actuator 50₂ to coupling strut 32 can be by any suitable medium, such as by epoxy or other adhesive, for example.

Preferably both first piezoelectric actuator 50₁ and second piezoelectric actuator 502 have a compliant periphery or complaint member, such as an O-ring 52, disposed about its periphery to facilitate selective contact with the interior wall of housing 22.

In an illustrated example embodiment, the first piezoelectric actuator 50₁ and the second piezoelectric actuator 50₂ can each acquire plural states in accordance with polarity and magnitude of an applied voltage. For instance, an example piezoelectric actuator can acquire or assume an essentially flat position at +300 positive volts; a slight crown or slight deflection at zero or at nominal volts; and, a significant or perhaps even greatest deflection at −100 volts.

Thus, in the example embodiment, these three states or positions of the piezoelectric actuator can be assumed or acquired in accordance with the activating signals (e.g., driving voltage) applied to each. The piezoelectric activation circuit 34(4) applies activating signals to first piezoelectric actuator 50₁ and second piezoelectric actuator 50₂ in a predetermined pattern to cause the piezoelectric actuator assembly 30(4)and the piston 28 coupled thereto to assume successively the five actuator assembly figurations respectively shown in FIG. 4A-FIG. 4E The five example actuator assembly configurations and the driving signals applied to the respective piezoelectric actuators comprising the piezoelectric assembly are shown in Table 1.

TABLE 1

| | | Driving signal | Driving signal |
|---|---|---|---|
| Piezoelectric | | applied to first | applied to first |
| Actuator Assembly | Figure illustrative | piezoelectric | piezoelectric |
| Configuration | of Configuration | actuator 50₁ | actuator 50₂ |
| 1 | | −100 volts | 0 volts |
| 2 | | −100 volts | +300 volts |
| 3 | | 0 volts | +300 volts |
| 4 | | 0 volts | −100 volts |
| 5 | | +300 volts | −100 volts |

In the first piezoelectric actuator assembly configuration of FIG. 4A, activation circuit 34(4) applies a −100V signal to first piezoelectric actuator 50₁ and a 0 V signal to second piezoelectric actuator 50₂. The signal applied to second electric actuator 50₂ causes second piezoelectric actuator 50₂ to assume its slightly crowned position or state, while the signal applied to first piezoelectric actuator 50₁ causes first piezoelectric actuator 50₁ to assume its significantly deflected state or position. As such, the periphery (e.g., O-ring 52) of second piezoelectric actuator 50₂ contacts and holds against the interior surface of housing 22, but first piezoelectric actuator 50₁ is not in contact with housing 22.

In the second piezoelectric actuator assembly configuration of FIG. 4B, piezoelectric activation circuit 34(4) continues application of the −100V signal to first piezoelectric actuator 50₁, but now applies a +300 V signal to second piezoelectric actuator 50₂. As such, first piezoelectric actuator 50₁ is still not in contact with housing 22. However, second piezoelectric actuator 50₂, while rigidly engaging the interior surface of housing 22, moves from its slightly crowned position to it essentially flat or straight position and thereby, by displacement of the center of second piezoelectric actuator 50₂, causes strut 32 and piston 28 affixed to second piezoelectric actuator 50₂ to displace or increment a distance 54_{B} toward orifice 26 as shown in FIG. 4B. Such movement causes fluid dispenser 20(4) to expel a unit or dosage of fluid from chamber 24 and through the orifice 26.

In the third piezoelectric actuator assembly configuration of FIG. 4C, piezoelectric activation circuit 34(4) continues application of the +300V signal to second piezoelectric actuator 50₂ but now applies a 0 V signal to first piezoelectric actuator 50₁. As such, first piezoelectric actuator 50₁ assumes its slightly crowned position and comes into contact with housing 22. Second piezoelectric actuator 50₂ remains essentially flat and continues to rigidly engaging the interior surface of housing 22.

In the fourth piezoelectric actuator assembly configuration of FIG. 4D, piezoelectric activation circuit 34(4) continues application of the 0V signal to first piezoelectric actuator 50₁, but now applies a −100 V signal to second piezoelectric actuator 50₂. As such, first piezoelectric actuator 50₁ remains in its slightly crowned position and in engaged contact with the interior surface of housing 22. However, second piezoelectric actuator 50₂ releases its contact with housing 22.

In the fifth piezoelectric actuator assembly configuration of FIG. 4E, piezoelectric activation circuit 34(4) continues application of the −100V signal to second piezoelectric actuator 50₂, but now applies a +300 V signal to first piezoelectric actuator 50₁. While rigidly engaging the interior surface of housing 22, first piezoelectric actuator 50₁ moves from its slightly crowned position to it essentially flat or straight position and thereby, by displacement of the center of first piezoelectric actuator 50₁, causes strut 32 and piston 28 affixed to first piezoelectric actuator 50₁ to displace or increment a distance ⁵⁴_{E} toward orifice 26 as shown in FIG. 4E. Such movement causes fluid dispenser 20(4) to expel another unit or dosage of fluid from chamber 24 and through the orifice 26. A sequence of further operations for expelling yet further doses is understood by repetition of the piezoelectric assembly configurations of FIG. 4A-FIG. 4E or other analogous and suitable configurations.

The piezoelectric actuator(s) comprising the piezoelectric actuator assemblies described herein can take the form of a multi-layered laminate (also known as a ruggedized laminated piezoelectric member). The multi-layered laminate can comprise a piezoelectric wafer which is laminated by an adhesive between a metallic substrate layer and an outer metal layer. Electrical leads for activating the piezoelectric wafer can be connected to electrodes which may be sputtered or otherwise formed on opposite sides of the piezoelectric wafer, or connected to the metallic substrate layer and outer metal layer. Example structures of the multi-layered piezoelectric laminate and processes for fabricating the same are described in or discernable from one or more of the following (all of which are incorporated herein by reference in their entirety): PCT Patent Application PCT/US01/28947, filed 14 Sep. 2001; U.S. patent application Ser. No. 10/380,547, filed Mar. 17, 2003, entitled “Piezoelectric Actuator and Pump Using Same”; U.S. patent application Ser. No. 10/380,589, filed Mar. 17, 2003; and U.S. Provisional Patent Application 60/670,692, filed Apr. 13, 2005, entitled “PIEZOELECTRIC DIAPHRAGM ASSEMBLY WITH CONDUCTORS ON FLEXIBLE FILM”, all of which are incorporated herein by reference.

Construction and operation of the piezoelectric activation circuits described herein is understood from drive electronics such as those described in U.S. patent application Ser. No. 10/816,000, filed Apr. 2, 2004 by Vogeley et al., entitled “Piezoelectric Devices and Methods and Circuits for Driving Same”, which is incorporated herein by reference in its entirety, or by documents referenced and/or incorporated by reference therein.

Fabrication of a piezoelectric actuator with an aperture or other central feature is understood with reference, e.g., to patent application Ser. No. 60/670,650, filed Apr. 13, 2005, entitled VALVING PIEZOELECTRIC DIAPHRAGM FOR PUMP, and U.S. patent application Ser. No. 11/104,667, filed Apr. 13, 2005, entitled ELECTRICALLY DRIVEN MECHANICAL ACTUATORS AND METHODS OF OPERATING SAME, both of which are incorporated herein by reference in their entirety.

Although various embodiments have been shown and described in detail, the claims are not limited to any particular embodiment or example. None of the above description should be read as implying that any particular element, step, range, or function is essential. It is to be understood that the invention is not to be limited to the disclosed embodiments, but on the contrary, is intended to cover various modifications and equivalent arrangements.

what is claimed is:

1. A fluid dispenser comprising:
a housing for defining an essentially cylindrical fluid chamber, the housing comprising an orifice;
a piston positioned in the housing for linear motion in the chamber for expelling fluid from the chamber and through the orifice;
a piezoelectric actuator assembly at least partially positioned in the housing for imparting the linear motion to the piston.

2. The apparatus of claim 1, wherein the piezoelectric actuator assembly comprises:
a first piezoelectric actuator;
a second piezoelectric actuator;
a circuit for applying activating signals to the first piezoelectric actuator and the second piezoelectric actuator whereby the first piezoelectric actuator and the second piezoelectric actuator transition between unactivated and activated positions for respectively contacting a wall of the housing and deflecting in a sequence whereby the piezoelectric actuator assembly and the piston coupled thereon linearly travel through the chamber for expelling fluid from the chamber and through the orifice.

3. The apparatus of claim 2, wherein the piezoelectric actuator assembly further comprises a coupling strut which is connected to a non-fluid contacting side of the piston, the strut extending in a direction along a major cylindrical axis of the fluid chamber, the strut having the first piezoelectric actuator and the second piezoelectric actuator attached thereto.

4. The apparatus of claim 3, wherein the strut extends through apertures formed in the first piezoelectric actuator and the second piezoelectric actuator.

5. The apparatus of claim 1, wherein the piezoelectric actuator assembly comprises a piezoelectric activation circuit and at least one piezoelectric actuator member, and wherein both the piezoelectric activation member and the piezoelectric activation circuit are situated within the housing.

6. The apparatus of claim 5, wherein housing comprises a lid, and wherein the piezoelectric activation circuit is carried by the lid.

7. The apparatus of claim 6, wherein the lid carries an activation switch for providing input to the piezoelectric activation circuit.

8. The apparatus of claim 6, wherein the lid carries an indicator for providing an indication of operation of the fluid dispenser.

9. The apparatus of claim 6, wherein the lid carries an indicator for providing an identification of the fluid dispenser.

10. The apparatus of claim 1, wherein the piezoelectric actuator assembly comprises a piezoelectric activation circuit and at least one piezoelectric actuator member, and wherein the piezoelectric activation circuit is external to the housing.

11. A syringe for dispensing a fluid comprising:
a syringe housing for defining an essentially cylindrical fluid chamber, the housing having an orifice;
a piston positioned in the housing for linear motion in the chamber for expelling fluid from the chamber and through the orifice;
a piezoelectric actuator assembly positioned in the housing for imparting the linear motion to the piston.

12. The apparatus of claim 11, wherein the piezoelectric actuator assembly comprises:
a first piezoelectric actuator;
a second piezoelectric actuator;
a circuit for applying activating signals to the first piezoelectric actuator and the second piezoelectric actuator whereby the first piezoelectric actuator and the second piezoelectric actuator transition between unactivated and activated positions for respectively contacting a wall of the housing and deflecting in a sequence whereby the piezoelectric actuator assembly and the piston coupled thereon linearly travel through the chamber for expelling fluid from the chamber and through the orifice.

13. The apparatus of claim 12, wherein the piezoelectric actuator assembly further comprises a coupling strut which is connected to a non-fluid contacting side of the piston, the strut extending in a direction along a major cylindrical axis of the fluid chamber, the strut having the first piezoelectric actuator and the second piezoelectric actuator attached thereto.

14. The apparatus of claim 13, wherein the strut extends through apertures formed in the first piezoelectric actuator and the second piezoelectric actuator.

15. The apparatus of claim 11, wherein the piezoelectric actuator assembly comprises a piezoelectric activation circuit and at least one piezoelectric actuator member, and wherein both the piezoelectric activation member and the piezoelectric activation circuit are situated within the housing.

16. The apparatus of claim 15, wherein housing comprises a lid, and wherein the piezoelectric activation circuit is carried by the lid.

17. The apparatus of claim 16, wherein the lid carries an activation switch for providing input to the piezoelectric activation circuit.

18. The apparatus of claim 16, wherein the lid carries an indicator for providing an indication of operation of the fluid dispenser.

19. The apparatus of claim 16, wherein the lid carries an indicator for providing an identification of the fluid dispenser.

20. The apparatus of claim 11, wherein the piezoelectric actuator assembly comprises a piezoelectric activation circuit and at least one piezoelectric actuator member, and wherein the piezoelectric activation circuit is external to the housing.
